(19) United States
(12) Patent Application Publication
(10) Pub. No.: US 2019/0059368 A1
(43) Pub. Date: Feb. 28, 2019

(54) 3,5-DIMETHOXYSTILBENE ANALOGS AND USES THEREOF

(71) Applicant: The United States of America, as represented by the Secretary of Agriculture, Washington, DC (US)

(72) Inventors: AGNES M. RIMANDO, OXFORD, MS (US); JIAN-QUAN WENG, UNIVERSITY, MS (US)

(21) Appl. No.: 16/056,601

(22) Filed: Aug. 7, 2018

Related U.S. Application Data
(60) Provisional application No. 62/559,905, filed on Sep. 18, 2017.
(60) Provisional application No. 62/552,501, filed on Aug. 31, 2017.

(51) Int. Cl. A01N 31/16; A01N 33/22; C07C 41/30; C07C 201/12

(52) U.S. Cl. CPC A01N 31/16; C07C 201/12; C07C 41/30; A01N 33/22

(57) ABSTRACT

Analogs of stilbene, and in particular, 3,5-dimethoxystilbene, are disclosed. Also disclosed are various uses for the different compounds described. The uses of the disclosed 3,5-dimethoxystilbene analogs include treatment as a pesticide, nematicide, fungicide, bactericide, and antimicrobial agent. Many of the analogs are novel, and procedures for synthesis are also provided.

### REFERENCE TO RELATED APPLICATIONS

This application claims the benefit of U.S. Provisional Application No. 62/552,501 filed Aug. 31, 2017, and U.S. Provisional Application No. 62/559,905 filed Sep. 18, 2017, both of which are incorporated herein by reference in their entireties.

### BACKGROUND

Stilbenes have been widely studied for their medicinal properties. In contrast, while some are known as phytoalexins, studies on their pesticidal activity have been sparse. Aedes aegypti is the primary vector for the transmission of dengue. More than 500,000 people are hospitalized each year due to dengue and about 20,000 cases lead to severe complications resulting in death. Dengue has become increasingly frequent in the United States, and this trend is also occurring worldwide. Ae. aegypti is also the vector for yellow fever, chikungunya, and other tropical diseases which cause severe human health problems throughout the world. There are no effective vaccines or drugs for the control of these diseases, and controlling the mosquito vectors remains the major strategy for disease prevention.

In addition, plant-parasitic nematodes cause crop losses of approximately $10 billion each year in the United States and $125 billion globally. Many of the conventional nematicides used to manage these plant pathogens have been deregistered due to adverse effects on health and the environment. It is therefore necessary to develop efficacious, environmentally safe means of managing phytoparasitic nematodes. One nematode to study is the genus Meloidogyne (root-knot nematode), which is one of the most economically important nematodes attacking crop plants; species in this genus are found worldwide on numerous hosts. Of specific interest is the southern root-knot nematode Meloidogyne incognita (, a highly destructive species in this genus.

Biologically active compounds may be of great use in various fields. Thus, it is of interest to synthesize and test new analogs of 3,5-dimethoxystilbene for different activities, and also to test known analogs for the same activities.

All of the references cited herein, including U.S. Patents and U.S. Patent Application Publications, are incorporated by reference in their entirety.

Mention of trade names or commercial products in this publication is solely for the purpose of providing specific information and does not imply recommendation or endorsement by the U.S. Department of Agriculture.

### SUMMARY

According to at least one aspect of the invention, a composition may include a stilbene analog, the stilbene analog being one of (Z)-1,3-dimethoxy-2-methyl-5-styrylbenzene (compound 7a), (E)-1,3-dimethoxy-5-(3-methoxystyryl)-2-methylbenzene (compound 6b), (Z)-1,3-dimethoxy-5-(3-methoxystyryl)-2-methylbenzene (compound 7b), (E)-1,3-dimethoxy-5-(4-methoxystyryl)-2-methylbenzene (compound 6c), (Z)-1,3-dimethoxy-5-(4-methoxystyryl)-2-methylbenzene (compound 7c), (E)-1,3-dimethoxy-5-(4-nitrostyryl)-2-methylbenzene (compound 6d), (Z)-1,3-dimethoxy-5-(4-nitrostyryl)-2-methylbenzene (compound 7d), (E)-1,3-dimethoxy-5-(4-fluoro styryl)-2-methylbenzene (compound 6e), (Z)-1,3-dimethoxy-5-(4-fluoro styryl)-2-methylbenzene (compound 7e), (E)-1,3-dimethoxy-5-(4-chlorostyryl)-2-methylbenzene (compound 6f), (Z)-1,3-dimethoxy-5-(4-chlorostyryl)-2-methylbenzene (compound 7f), (E)-1,3-dimethoxy-5-(4-bromostyryl)-2-methylbenzene (compound 6g), (Z)-1,3-dimethoxy-5-(4-bromostyryl)-2-methylbenzene (compound 7g), (E)-1,3-dimethoxy-5-(4-trifluoromethylstyryl)-2-methylbenzene (compound 6h), (Z)-1,3-dimethoxy-5-(4-trifluoromethylstyryl)-2-methylbenzene (compound 7h), (E)-1,3-dimethoxy-5-(3,4-dichlorostyryl)-2-methylbenzene (compound 6i), (Z)-1,3-dimethoxy-5-(3,4-dichlorostyryl)-2-methylbenzene (compound 7i), (E)-1,3-dimethoxy-5-(2,4-dimethoxystyryl)-2-methylbenzene (compound 6j), (Z)-1,3-dimethoxy-5-(2,4-dimethoxystyryl)-2-methylbenzene (compound 7j), (E)-1,3-dimethoxy-5-(3,4-dimethoxystyryl)-2-methylbenzene (compound 6k), (Z)-1,3-dimethoxy-5-(3,4-dimethoxystyryl)-2-methylbenzene (compound 7k), (E)-1,3-dimethoxy-5-(3,5-dimethoxystyryl)-2-methylbenzene (compound 6l), (Z)-1,3-dimethoxy-5-(3,5-dimethoxystyryl)-2-methylbenzene (compound 7l), (E)-1,3-dimethoxy-5-(2,6-dimethoxy-4-methylstyryl)-2-methylbenzene (compound 6m), (Z)-1,3-dimethoxy-5-(2,6-dimethoxy-4-methylstyryl)-2-methylbenzene (compound 7m), (Z)-4-(3,5-dimethoxystyryl)-2-(3-methylbut-2-en-1-yl)phenol (compound 12), (E)-4-(3,5-dimethoxy-4-methylstyryl)phenol (compound 16), and (Z)-4-(3,5-dimethoxy-4-methylstyryl)phenol (compound 15).

According to a further aspect of the invention, the composition may have biological activity.

According to a further aspect of the invention, the biological activity may be at least one of insecticidal activity, larvicidal activity, fungicidal activity, nematicidal activity, antimicrobial activity, antibiotic activity, and bactericidal activity.

According to a further aspect of the invention, the composition may include a carrier.

According to a further aspect of the invention, the stilbene analog may be one of compounds 7a, 7e, 6f, 6g, 6i, 7k, 12, 15, and 16.

According to another aspect of the invention, a method of treating for mosquitos may include applying an effective amount of a pesticide to a plant or area, the pesticide being an analog of 3,5-dimethoxystilbene.

According to a further aspect of the invention, the pesticide may have a prenyl group substitution in the 3-position of the non-dimethoxy-substituted benzene ring.

According to a further aspect of the invention, the pesticide may be one of (E)-4-(3,5-dimethoxystyryl)-2-(3-methylbut-2-en-1-yl)phenol (compound 11) and (Z)-4-(3,5-dimethoxystyryl)-2-(3-methylbut-2-en-1-yl)phenol (compound 12).

According to a further aspect, the mosquitos may be Aedes sp.

According to still a further aspect, the mosquitos may be Ae. aegypti.

According to another aspect of the invention, a method of treating for nematodes may include applying an effective amount of a nematicide to a plant or area, the nematicide being an analog of 3,5-dimethoxystilbene.

According to a further aspect of the invention, the nematicide may be one of (E)-1,3-dimethoxy-5-(4-chlorostyryl)-2-methylbenzene (compound 6f), (E)-1,3-dimethoxy-5-(4-bromostyryl)-2-methylbenzene (compound 6g), (E)-1,3-dimethoxy-5-(3,4-dichlorostyryl)-2-methylbenzene (compound 6i), (Z)-1,3-dimethoxy-5-(3,4-dimethoxystyryl)-2-methylbenzene (compound 7k), and (E)-5-(4-hydroxystyryl)-2-methylbenzene-1,3-diol (compound 17).

According to a further aspect, the nematodes may be Meloidogyne sp.

According to still a further aspect, the nematodes may be M. incognita.

According to another aspect of the invention, a method of treating for fungi may include applying an effective amount of a fungicide to a plant or area, the fungicide being an analog of 3,5-dimethoxystilbene.

According to a further aspect of the invention, the fungicide may be one of (E)-1,3-dimethoxy-2-methyl-5-styrylbenzene (compound 6a), (Z)-1,3-dimethoxy-2-methyl-5-styrylbenzene (compound 7a), and (Z)-1,3-dimethoxy-5-(4-fluorostyryl)-2-methylbenzene (compound 7e).

According to a further aspect, the fungi may be Colletotrichum sp.

According to still a further aspect, the fungi may be one of C. acutatum, C. fragariae, and C. gloeosporioides.

According to another aspect of the invention, a method of treating for a human pathogen may include providing an effective amount of an active compound, the active compound being an analog of 3,5-dimethoxystilbene, and the active compound having a hydroxy group substitution in the 4-position of the non-dimethoxy-substituted benzene ring.

According to a further aspect of the invention, the active compound may have a prenyl group substitution in the 3-position of the non-dimethoxy-substituted benzene ring.

According to a further aspect of the invention, the active compound may be one of (E)-4-(3,5-dimethoxystyryl)-2-(3-methylbut-2-en-1-yl)phenol (compound 11), (Z)-4-(3,5-dimethoxystyryl)-2-(3-methylbut-2-en-1-yl)phenol (compound 12), (Z)-4-(3,5-dimethoxy-4-methylstyryl)phenol (compound 15), (E)-4-(3,5-dimethoxy-4-methylstyryl)phenol (compound 16), and (E)-5-(4-hydroxystyryl)-2-methylbenzene-1,3-diol (compound 17).

According to a further aspect, the human pathogen may be one of Cryptococcus neoformans, Staphylococcus aureus, and Mycobacterium intracellulare.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

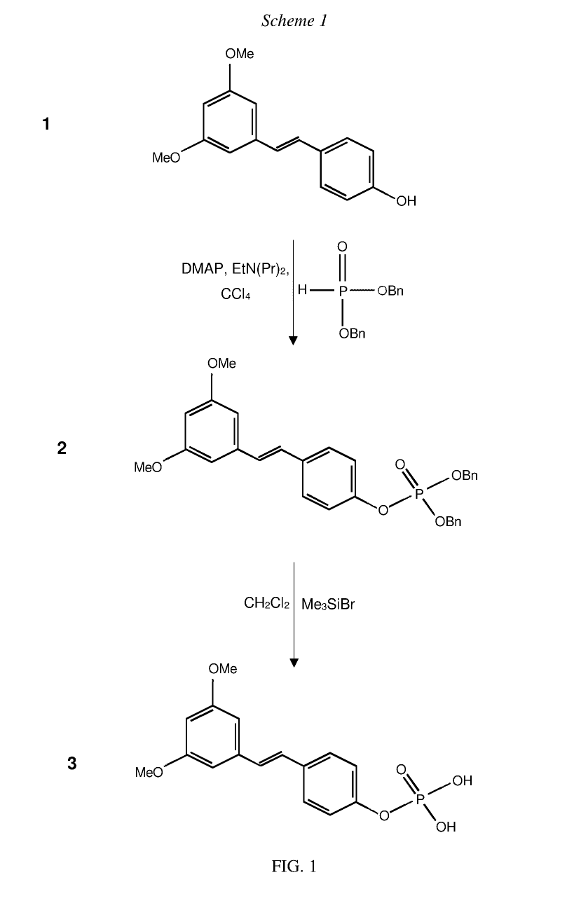

Exemplary FIG. 1 shows an exemplary scheme (“Scheme 1”) for producing stilbene analogs according to the present invention.

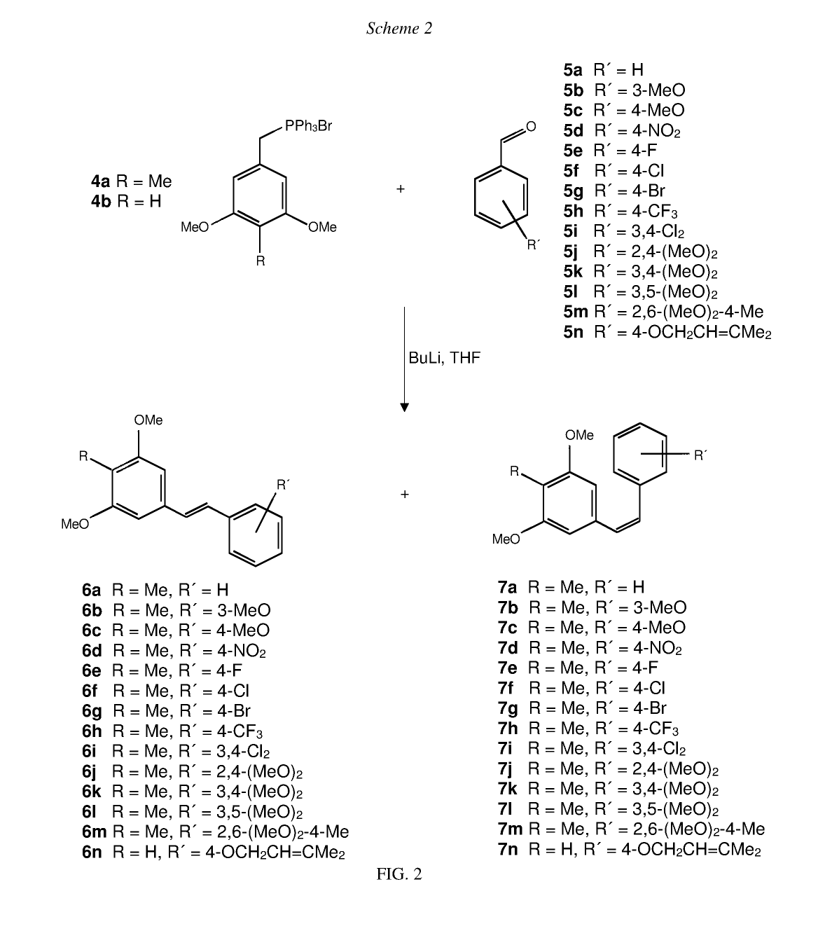

Exemplary FIG. 2 shows an exemplary scheme (“Scheme 2”) for producing stilbene analogs according to the present invention.

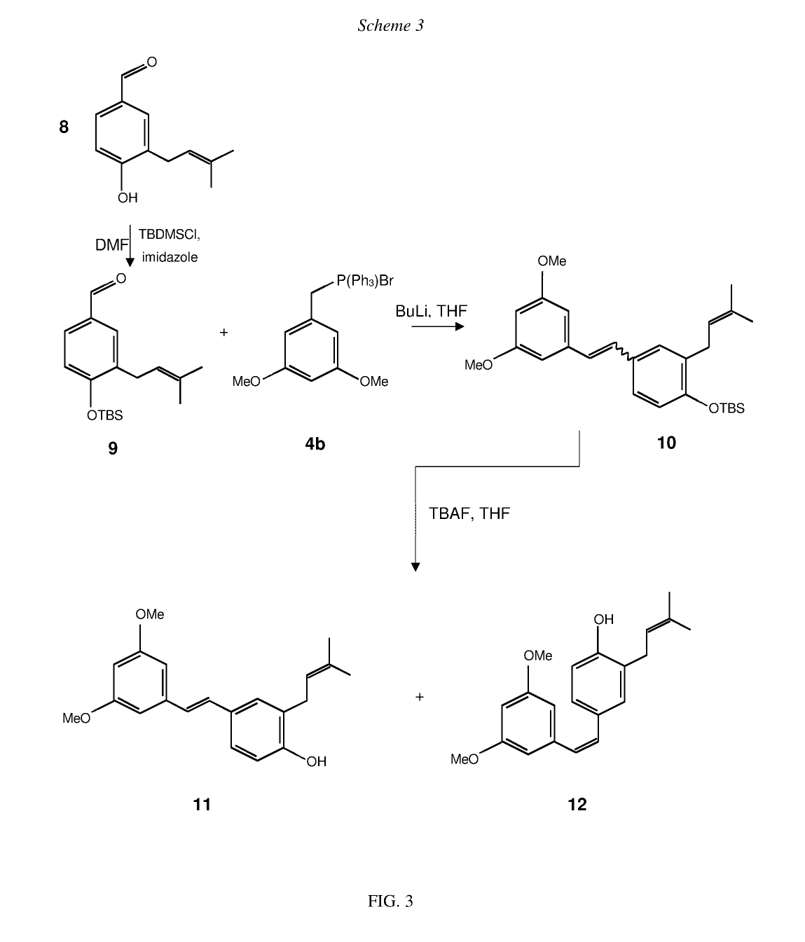

Exemplary FIG. 3 shows an exemplary scheme (“Scheme 3”) for producing stilbene analogs according to the present invention.

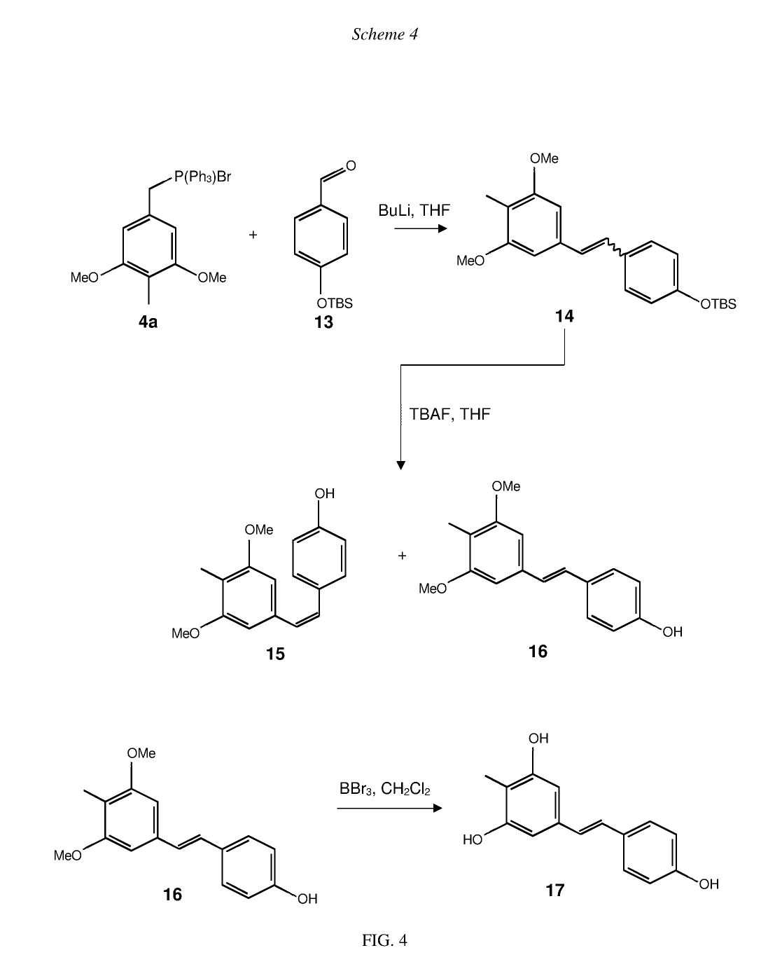

Exemplary FIG. 4 shows an exemplary scheme (“Scheme 4”) for producing stilbene analogs according to the present invention.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

Unless defined otherwise, all technical and scientific terms used herein have the same meaning as commonly understood by one of ordinary skill in the art to which the invention belongs. As used herein, the term “about” refers to a quantity, level, value, or amount that varies by as much as 30%, preferably by as much as 20%, and more preferably by as much as 10% to a reference quantity, level, value, or amount. Although any methods and materials similar or equivalent to those described herein can be used in the practice or testing of the present invention, the preferred methods and materials are now described.

Other compounds may be added to the composition provided they do not substantially interfere with the intended activity and efficacy of the composition; whether or not a compound interferes with activity and/or efficacy can be determined, for example, by the procedures utilized below.

The amounts, percentages, and ranges disclosed herein are not meant to be limiting, and increments between the recited amounts, percentages, and ranges are specifically envisioned as part of the invention.

“Biological activity” refers to the ability of a compound or composition to (i) prevent the growth of a pest or pathogen, (ii) inhibit the growth of a pest or pathogen, or (ii) substantially kill or eliminate a pest or pathogen population. A biological active is a compound or composition which exhibits substantial biological activity.

The term “treat,” “treating,” or “treatment,” as used herein, refers to the use of a composition to reduce or prevent a condition, symptom, or disease caused by a pathogen by (i) preventing the growth of the pathogen, (ii) inhibiting the growth of the pathogen or its sporulation, or (ii) substantially killing or eliminating the pathogen. In addition, the term “treat,” “treating,” or “treatment” may also refer to the use of a composition to kill, reduce the population of, or inhibit the growth of a pest or pest population.

The term “effective amount” of a compound or property as provided herein is meant such amount as is capable of performing the function of the compound or property for which an effective amount is expressed. As will be pointed out below, the exact amount required will vary from process to process, depending on recognized variables such as the compounds employed and the processing conditions observed. Thus, it is not possible to specify an exact “effective amount.” However, an appropriate effective amount may be determined by one of ordinary skill in the art using only routine experimentation.

As used herein, “analog” or “chemical variant” of a compound refers to a structural analog of the identified compound having a similar structure and similar activity.

The term “consisting essentially of” excludes additional method steps or composition components that substantially interfere with the intended activity of the method or composition, and can be readily determined by those skilled in the art (for example, from a consideration of this specification or practice of the invention disclosed herein).

The invention illustratively disclosed herein suitably may be practiced in the absence of any element (e.g., method steps or composition components) which is not specifically disclosed herein.

According to at least one exemplary embodiment, a stilbene analog may have biological activity. Further, the stilbene analog may be an analog of 3,5-dimethoxystilbene. The stilbene analog may have activity, for example as an insecticide, larvicide, fungicide, nematicide, antimicrobial agent, antibiotic, and/or bactericide.

The stilbene analog may be one of several analogs of 3,5-dimethoxystilbene. Without being limited to the following examples, the analog may be (E)-4-(3,5-dimethoxystyryl)phenyl dihydrogen phosphate (compound 3), (E)-1,3-dimethoxy-2-methyl-5-styrylbenzene (compound 6a), (Z)-1,3-dimethoxy-2-methyl-5-styrylbenzene (compound 7a), (E)-1,3-dimethoxy-5-(3-methoxystyryl)-2-methylbenzene (compound 6b), (Z)-1,3-dimethoxy-5-(3-methoxystyryl)-2-methylbenzene (compound 7b), (E)-1,3-dimethoxy-5-(4-methoxystyryl)-2-methylbenzene (compound 6c), (Z)-1,3-dimethoxy-5-(4-methoxystyryl)-2-methylbenzene (compound 7c), (E)-1,3-dimethoxy-5-(4-nitrostyryl)-2-methylbenzene (compound 6d), (Z)-1,3-dimethoxy-5-(4-nitrostyryl)-2-methylbenzene (compound 7d), (E)-1,3-dimethoxy-5-(4-fluorostyryl)-2-methylbenzene (compound 6e), (Z)-1,3-dimethoxy-5-(4-fluorostyryl)-2-methylbenzene (compound 7e), (E)-1,3-dimethoxy-5-(4-chlorostyryl)-2-methylbenzene (compound 6f), (Z)-1,3-dimethoxy-5-(4-chlorostyryl)-2-methylbenzene (compound 7f), (E)-1,3-dimethoxy-5-(4-bromostyryl)-2-methylbenzene (compound 6g), (Z)-1,3-dimethoxy-5-(4-bromostyryl)-2-methylbenzene (compound 7g), (E)-1,3-dimethoxy-5-(4-trifluoromethylstyryl)-2-methylbenzene (compound 6h), (Z)-1,3-dimethoxy-5-(4-trifluoromethylstyryl)-2-methylbenzene (compound 7h), (E)-1,3-dimethoxy-5-(3,4-dichlorostyryl)-2-methylbenzene (compound 6i), (Z)-1,3-dimethoxy-5-(3,4-dichlorostyryl)-2-methylbenzene (compound 7i), (E)-1,3-dimethoxy-5-(2,4-dimethoxystyryl)-2-methylbenzene (compound 6j), (Z)-1,3-dimethoxy-5-(2,4-dimethoxystyryl)-2-methylbenzene (compound 7j), (E)-1,3-dimethoxy-5-(3,4-dimethoxystyryl)-2-methylbenzene (compound 6k), (Z)-1,3-dimethoxy-5-(3,4-dimethoxystyryl)-2-methylbenzene (compound 7k), (E)-1,3-dimethoxy-5-(3,5-dimethoxystyryl)-2-methylbenzene (compound 6I), (Z)-1,3-dimethoxy-5-(3,5-dimethoxystyryl)-2-methylbenzene (compound 7l), (E)-1,3-dimethoxy-5-(2,6-dimethoxy-4-methylstyryl)-2-methylbenzene (compound 6m), (Z)-1,3-dimethoxy-5-(2,6-dimethoxy-4-methylstyryl)-2-methylbenzene (compound 7m), (E)-1,3-dimethoxy-5-(4-((3-methylbut-2-en-1-yl)oxy)styryl)benzene (compound 6n), (Z)-1,3-dimethoxy-5-(4-((3-methylbut-2-en-1-yl)oxy)styryl)benzene (compound 7n), (E)-4-(3,5-dimethoxystyryl)-2-(3-methylbut-2-en-1-yl)phenol (compound 11), (Z)-4-(3,5-dimethoxystyryl)-2-(3-methylbut-2-en-1-yl)phenol (compound 12), (E)-4-(3,5-dimethoxy-4-methylstyryl)phenol (compound 16), (Z)-4-(3,5-dimethoxy-4-methylstyryl)phenol (compound 15), and (E)-5-(4-hydroxystyryl)-2-methylbenzene-1,3-diol (compound 17).

The stilbene analog may be present by itself or in the presence of a carrier, for example a solvent.

Analogs of the present invention may be synthesized according to one of the exemplary schemes shown in FIGS. 1-4. An analog of 3,5-dimethoxystilbene according to the present invention can be tested for biological activity according to known methods in the art.

### Synthesis of 3,5-Dimethoxystilbene Analogs

Several analogs of 3,5-dimethoxystilbene were synthesized for testing of biological activity. All solvents were redistilled before use. All reactions were performed in a dry round-bottom flask and occurred under N₂ atmosphere. Reactions were monitored by thin-layer chromatography (TLC) using a TLC SiO₂ 60 F₂₅₄ (SiO₂; Merck); the spots were visualized under UV light. Purification was performed by prep. TLC on SiO₂ GF plates (SORBTECH, scored, 20×20 cm, 500 μm) or flash chromatography using SiO₂ (40−60 μm; Sorbent Technologies). NMR Spectra were recorded on a Bruker Avance DRX-500 MHz spectrometer in CDCl₃, (D₆) DMSO, or (D₆)acetone. ESI-MS spectra were collected using a JEOL AccuTOF JMS-T100LC mass spectrometer (JEOL Inc., Peabody, Mass., USA). GC/MS Spectra were obtained with a JEOL GCMate II spectrometer coupled with an Agilent 6890N gas chromatograph (Agilent Technologies, Santa Clara, Calif., USA).

For the synthesis of compounds 6a-6n and 7a-7n, BuLi (1.6M in hexanes, 1.0 equiv.) was added to a cold soln. (−78° C.) of phosphonium salt (1.0 equiv.) in THF, and the resulting red soln. was stirred under N₂ for 2 h. A soln. of aldehyde (1.0 equiv.) in THF was added dropwise over 30 min, and the mixture was stirred for 12 h at room temperature (r.t.) (FIG. 2). The resulting suspension was poured into H₂O and extracted with CH₂Cl₂ multiple times. The combined organic phase was washed with brine and dried (MgSO₄). After solvent removal under reduced pressure, the crude product was purified by prep. TLC or flash chromatography. The (Z)-isomer eluted first followed by the (E)-isomer.

Further details of the synthesized analogs beyond the synthesis described below can be found in “Synthesis and Biological Evaluation of 3,5-Dimethoxystilbene Analogs” by Weng, et al. (Chem. Biodiversity 2016, 13, 1165-1177), the contents of which are hereby incorporated herein in their entirety.

(E)-4-(3,5-dimethoxystyryl)phenyl dihydrogen phosphate (compound 3). Compound 3 was synthesized through reaction of pterostilbene (1) and dibenzylphosphonate using 4-(dimethylamino)pyridine (DMAP) and EtN(¹Pr)₂, followed by deprotection of the Bn groups (Figue 1).

Pterostilbene, (3,5-dimethoxybenzyl)(triphenyl)phosphonium bromide (compound 4b), and 4-{[tert-butyl(dimethyl)silyl]oxy}benzaldehyde (compound 13) were synthesized according to published methods.

(3,5-Dimethoxy-4-methylbenzyl)(triphenyl)phosphonium bromide (compound 4a). To a soln. of 5-(bromomethyl)-1,3-dimethoxy-2-methylbenzene (500 mg, 1.938 mmol) in toluene (10 ml) was added Ph₃P (565.4 mg, 2.134 mmol). The soln. was heated at reflux for 6 h. The resulting precipitate was collected and recrystallized from EtOH as a white solid (930.0 mg, 94.5%).

4-{[tert-Butyl(dimethyl)silyl]oxy}-3-(3-methylbut-2-en-1-yl)benzaldehyde (compound 9). To a soln. of 4-hydroxy-3-(3-methylbut-2-en-1-yl)benzaldehyde (8; 200 mg, 1.0 mmol) was added imidazole (96.3 mg, 1.4 mmol) in DMF (10 ml) and tert-butyl(dimethyl)silyl chloride (202 mg, 1.3 mmol). The soln. was left stirring for 20 h at r.t., and then the mixture was poured into H₂O and extracted with AcOEt. The organic phase was combined and dried (MgSO₄), and the solvent was removed under reduced pressure. The crude product was purified by flash chromatography (hexanes/AcOEt 95:5) to give 9 (283.2 mg, 90.3%) as a viscous liquid.

1,3-dimethoxy-2-methyl-5-styrylbenzene (compounds 6a and 7a). (3,5-Dimethoxy-4-methylbenzyl)(triphenyl)phosphonium bromide (4a; 200 mg, 0.394 mmol) was reacted with benzaldehyde (5a; 42.3 mg, 0.394 mmol), then purified by prep. TLC (hexanes/AcOEt 97:3) to afford 6a and 7a. Compound 6a was obtained as a white solid: 22.4 mg (22.3%). Compound 7a was obtained as a viscous liquid: 57.8 mg (57.7%).

1,3-dimethoxy-5-(3-methoxystyryl)-2-methylbenzene (compounds 6b and 7b). Compound 4a (200 mg, 0.394 mmol) was reacted with 3-methoxybenzaldehyde (5b; 55.3 mg, 0.394 mmol), then purified by prep. TLC (hexanes/AcOEt 95:5) to afford 6b and 7b. Compound 6b was obtained as a white solid: 34.9 mg (31.1%). Compound 7b was obtained as a viscous liquid: 69.3 mg (61.8%).

1,3-dimethoxy-5-(4-methoxystyryl)-2-methylbenzene (compounds 6c and 7c). Compound 4a (200 mg, 0,394 mmol) was reacted with 4-methoxybenzaldehyde (5c; 54.8 mg, 0.394 mmol), then purified by prep. TLC (hexanes/AcOEt 95:5) to afford 6c and 7c. Compound 6c was obtained as a white solid: 25.3 mg (22.6%). Compound 7c was obtained as a viscous liquid: 41.5 mg (37.0%).

1,3-dimethoxy-5-(4-nitrostyryl)-2-methylbenzene (compounds 6d and 7d). Compound 4a (200 mg, 0.394 mmol) was reacted with 4-nitrobenzaldehyde (5d; 60.8 mg, 0.394 mmol), then purified by prep. TLC (hexanes/AcOEt 95:5) to afford 6d and 7d. Compound 6d was obtained as a yellow solid: 19.0 mg (16.1%). Compound 7d was obtained as a yellow solid: 76.0 mg (64.4%).

1,3-dimethoxy-5-(4-fluorostyryl)-2-methylbenzene (compounds 6e and 7e). Compound 4a (200 mg, 0.394 mmol) was reacted with 4-fluorobenzaldehyde (5e; 49.9 mg, 0.394 mmol), then purified by prep. TLC (hexanes/AcOEt 98:2) to afford 6e and 7e. Compound 6e was obtained as a white solid: 21.8 mg (20.3%). Compound 7e was obtained as a viscous liquid: 29.1 mg (27.1%)

1,3-dimethoxy-5-(4-chlorostyryl)-2-methylbenzene (compound 6f and 7f). Compound 4a (200 mg, 0.394 mmol) was reacted with 4-chlorobenzaldehyde (5f; 56.3 mg, 0.394 mmol), then purified by prep. TLC (hexanes/AcOEt 98:2) to afford 6f and 7f. Compound 6f was obtained as a white solid: 41.2 mg (36.2%). Compound 7f was obtained as a viscous liquid: 36.2 mg (32.0%).

1,3-dimethoxy-5-(4-bromostyryl)-2-methylbenzene (compounds 6g and 7g). Compound 4a (200 mg, 0.394 mmol) was reacted with 4-bromobenzaldehyde (5g; 73.7 mg, 0.394 mmol), then purified by prep. TLC (hexanes/AcOEt 98:2) to afford 6g and 7g. Compound 6g was obtained as a white solid: 26.2 mg (19.9%). Compound 7g was obtained as a viscous liquid: 41.8 mg (31.8%).

1,3-dimethoxy-5-(4-trifluoromethylstyryl)-2-methylbenzene (compounds 6h and 7h). Compound 4a (200 mg, 0.394 mmol) was reacted with 4-(trifluoromethyl)benzaldehyde (5h; 72.3 mg, 0.394 mmol), then purified by prep. TLC (hexanes/acetone 95:5) to afford 6h and 7h. Compound 6h was obtained as a white solid: 49.4 mg (38.9%). Compound 7h was obtained as a viscous liquid: 51.8 mg (40.7%).

1,3-dimethoxy-5-(3,4-dichlorostyryl)-2-methylbenzene (compounds 6i and 7i). Compound 4a (200 mg, 0.394 mmol) was reacted with 3,4-dichlobenzaldehyde (5i; 70.4 mg, 0.394 mmol), then purified by prep. TLC (hexanes/acetone, 95:5) to afford 6i and 7i. Compound 6i was obtained as a white solid: 43.0 mg (33.7%). Compound 7i was obtained as a viscous liquid: 46.8 mg (36.7%).

1,3-dimethoxy-5-(2,4-dimethoxystyryl)-2-methylbenzene (compounds 6j and 7j). Compound 4a (200 mg, 0.394 mmol) was reacted with 2,4-dimethoxybenzaldehyde (5j; 66.8 mg, 0.394 mmol), then purified by prep. TLC (hexanes/AcOEt 90:10) to afford 6j and 7j. Compound 6j was obtained as a white solid: 31.2 mg (25.2%). Compound 7j was obtained as a white solid: 46.0 mg (37.1%).

1,3-dimethoxy-5-(3,4-dimethoxystyryl)-2-methylbenzene (compounds 6k and 7k). Compound 4a (200 mg, 0.394 mmol) was reacted with 3,4-dimethoxybenzaldehyde (5k; 66.2 mg, 0.394 mmol), then purified by prep. TLC (hexanes/AcOEt 80:20) to afford 6k and 7k. Compound 6k was obtained as a white solid: 48.3 mg (38.9%). Compound 7k was obtained as a white solid: 60.2 mg (48.6%).

1,3-dimethoxy-5-(3,5-dimethoxystyryl)-2-methylbenzene (compounds 6l and 7l). Compound 4a (200 mg, 0.394 mmol) was reacted with 3,5-dimethoxybenzalde-hyde (5l; 65.5 mg, 0.394 mmol) then purified by prep. TLC (hexanes/AcOEt 90:10) to afford 6l and 7l. Compound 6l was obtained as a white solid: 26.0 mg (20.9%). Compound 7l was obtained as a white solid: 46.0 mg (37.1%).

1,3-dimethoxy-5-(2,6-dimethoxy-4-methylstyryl)-2-methylbenzene (compounds 6m and 7m). Compound 4a (200 mg, 0.394 mmol) was reacted with 2,6-dimethoxy-4-methylbenzaldehyde (5m, 73.2 mg, 0.394mmol), then purified by prep. TLC (hexanes/acetone 90:10) to afford 6m and 7m. Compound 6m was obtained as a white solid: 20.5 mg (15.8%). Compound 7m was obtained as a white solid: 73.5 mg (56.8%).

1,3-dimethoxy-5-(4-((3-methylbut-2-en-1-yl)oxy)styryl)benzene (compounds 6n and 7n). (3,5-Dimethoxybenzyl)(triphenyl)phosphonium (4b; 379.5 mg, 0.769 mmol) was reacted with 4-[(3-methylbut-2-en-1-yl)oxy]benzaldehyde (5n; 154.0 mg, 0.769 mmol), then purified by prep. TLC (hexanes/AcOEt 97:3) to afford 6n and 7n. Compound 6n was obtained as a white solid: 56.4 mg (22.6%). Compound 7n was obtained as a viscous liquid: 163.2 mg (65.4%)

4-(3,5-dimethoxystyryl)-2-(3-methylbut-2-en-1-yl)phenol (compounds 11 and 12). Compound 4b (453.9 mg, 0.92 mmol) was reacted with 4-{[tert-butyl(dimethyl)silyl]oxy}-3-(3-methylbut-2-en-1-yl)benzaldehyde (9; 280 mg, 0.92 mmol), then purified by flash chromatography (hexanes/AcOEt 97:3) to afford 270 mg (66.9% yield) of a mixture of (Z)- and (E)-stilbene 10 (FIG. 3). Due to difficulty encountered in separating the two isomers, deprotection of TBS group was performed without isolation of the isomers. To a soln. of compound 10 (270 mg, 0.61 mmol in anh. THF) was added tetrabutylammmonium fluoride (TBAF; 800 μl, 0.80 mmol). The soln. was stirred for 45 min, poured into H₂O, extracted with CH₂Cl₂, and dried (MgSO₄). The solvent was removed under reduced pressure, and the crude product was purified by prep. TLC (hexanes/AcOEt 80:20) to afford 11 and 12. Compound 11 was obtained as a viscous liquid: 84.7 mg (42.4%). Compound 12 was obtained as a viscous liquid: 35.8 mg (17.9%).

4-(3,5-dimethoxy-4-methylstyryl)phenol (compounds 15 and 16). Compound 4a (326.5 mg, 0.643 mmol) was reacted with 4-{[tert-butyl(dimethyl)silyl]oxy}benzaldehyde (13; 152.0 mg, 0.643 mmol), then purified by flash chromatography (hexanes/AcOEt 97:3) to afford 185.7 mg (75.0% yield) of a mixture of (Z)-and (E)-stilbene 14 (FIG. 4). To a soln. of compound 14 (185.7 mg, 0.483 mmol in THF) was added TBAF (628 μl, 0.628 mmol). The soln. was stirred for 45 min, poured into H₂O, and extracted with CH₂Cl₂. The solvent was removed under reduced pressure, and the crude product was purified by prep. TLCs (hexanes/AcOEt 80:20) to afford 15 and 16. Compound 16 was obtained as a light-yellow solid (98.2 mg, 75.2%).

(E)-5-(4-hydroxystyryl)-2-methylbenzene-1,3-diol (compound 17). To a cold solution (−20° C.) of compound 16 (30 mg, 0.111 mmol in anh. CH₂Cl₂) was added dropwise BBr₃ (153 mg, 0.611 mmol; FIG. 4). The mixture was allowed to warm to r.t., and was stirred for 10 h. The reaction was quenched by ice water, and extracted with AcOEt. The organic phase was washed with H₂O and dried (Na₂SO₄). After removal of the solvent under reduced pressure, the crude product was purified by prep. TLC (hexanes/AcOEt 60:40) to afford compound 17 (9.0 mg, 33.5%) as a white solid.

### EXAMPLE 1

### Larvicidal Activity: Aedes aegypti

Analogs were screened for activity against Ae. aegypti larvae following a bioassay system described in Ali, A., et al. (J. Med. Entomol. 2014, 51, 824). Ae. aegypti larvae used in these studies were from a laboratory colony maintained at the Mosquito and Fly Research Unit at the Center for Medical, Agricultural and Veterinary Entomology, USDA-ARS, Gainesville, Fla. For larval bioassays, the eggs were hatched and the larvae were maintained at a temperature of 27±2° C. and 60±10% RH with a photoperiod regimen of 12:12 hour (Light:Dark). Five 1-day-old Ae. aegypti larvae were added into a droplet of H₂O to each well of 24-well plates (BD Labware, Franklin Lakes, N.J., USA) by use of a disposable 22.5-cm Pasteur pipette. A quantity of 50 μl of larval diet (2% slurry of 3:2 beef liver powder (Now Foods, Bloomingdale, Ill., USA) and Brewer's yeast (Lewis Laboratories Ltd., Westport, Conn., USA) was added to each well using a Finnpipette stepper (Thermo Fisher, Vantaa, Finland). All chemicals tested were diluted in DMSO. A quantity of 11 μl of the test chemical was added to the labeled wells, while 11 μl of DMSO was added to control treatments. After the treatment application, the plates were swirled in clockwise and counterclockwise motions and front and back and side to side five times to ensure even mixing of the chemicals. Larval mortality was recorded 24 hours post-treatment. Larvae that showed no movement in the well after manual disturbance of the water were recorded as dead. Three dosages, 100, 50, and 25 ppm, were used in the screening bioassay to determine the larvicidal activity and each treatment was replicated twice. A series of four dosages ranging between 50 and 6.25 ppm was used to determine the dose—response of 11 and 12, which showed high activity in the screening bioassay. Each treatment was replicated ten times. LC₅₀ Values for larvicidal data were calculated using SAS, Proc Probit. Control mortality was corrected using the Abbott's formula.

The results of the tests are shown in Table 1 below:

TABLE 1
Toxicity of compounds 11 and 12 against 1-day-old Ae. aegypti larvae

| Compound | LD₅₀ (95% CI)^{a} | LD₉₀ (95% CI)^{a} | χ² | df |
|---|---|---|---|---|
| 11 | 14.7^{b} | 34.7 | 72.4 | 38 |
| 12 | 16.2^{b} | 37.9 | 74.7 | 38 |

^{a}LD₅₀ and LD₉₀ are given in ppm at 95% confidence interval
^{b}14.7 ppm of 11 = 45.31 μM; 16.2 ppm of 12 = 49.93 μM

Of all of the analogs tested, only compounds 11 and 12 exhibited high larvicidal activity, with LC₅₀ values of 45.31 and 49.93 μM, respectively. Compounds 11 and 12 bear a prenyl substituent at C(3′) in the non-dimethoxy-substituted ring, differing from 6n and 7n where the prenyl group is in an ether linkage at C(4′) (See FIGS. 2 and 3). None of the other types of substituents (phosphate, MeO, halogen, NO₂, OH) appeared to confer larvicidal activity. The results suggest that a prenyl group directly attached to the benzene ring at C(3′) enhances larvicidal activity. It is noted that in the study of Ioset et al. (J. Nat. Prod. 2001, 64, 710), the prenylated stilbene derivatives tested for larvicidal activity had the prenyl group(s) in the other ring; these derivatives were less larvicidal than 3,5-dimethoxystilbene.

### EXAMPLE 2

### Larvicidal and Pesticidal Activity: Aedes aegypti

Further tests were performed on compounds 11 and 12 to determine their effect on larvae and adult mosquitoes. The compounds were tested in the standard bioassays using the Orlando (ORL), pesticide-susceptible strain and the Puerto Rico (PR), pyrethroid-resistant strain of Ae. aegypti. The ORL strain has been in continuous colony since 1952 with no pesticide exposure while the resistant PR strain was colonized from egg papers collected near San Juan, PR in June, 2012. The PR strain is available as a resistant reference strain through BEIResources/CDC. Mortality was determined in the larval assays at four different concentrations (1, 0.5, 0.25, and 0.1 μg/μl) and three concentrations for adults (6.25%, 3.125%, and 1.56%). Mortality was recorded 24 h post-application, and in all assays, a negative solvent and a positive control using permethrin were utilized; tests were done in triplicate. Assays were conducted according to published procedures as described in Wanner, J. et al. (Curr. Bioact. Compd. 2015, 11, 13).

The results of these tests are shown in Table 2 (larvae studies) and Table 3 (adult studies) below:

TABLE 2
Activity of compounds 11 and 12 against larvae
of ORL and PR strains of Ae. aegypti

| | | Percent Mortality^{a} | | | |
|---|---|---|---|---|---|
| | | Compound concentration (μg/μl) | | | |
| Compound | Strain | 1.0 | 0.5 | 0.25 | 0.1^{b} |
| 11^{c} | ORL | 100 | 100 | 100 | 100 |
| | PR | 100 | 100 | 100 | 93.3 ± 11.5 |
| 12^{d} | ORL | 93.3 ± 11.5 | 93.3 ± 11.5 | 93.3 ± 11.5 | 66.7 ± 23.1 |
| | PR | 93.3 ± 11.5 | 93.3 ± 11.5 | 73.3 ± 11.5 | 73.3 ± 11.5 |
| Permethrin | ORL | — | — | — | 100 |
| | PR | — | — | — | 93.3 ± 11.5 |

| | | |
|---|---|---|
| Control | ORL | 0 |
| (DMSO) | PR | 6.7 ± 11.5 |

^{a}Values are percent mortality ± standard deviation for mosquito larvae after 24 h
^{b}0.1 μg/μl of compounds 11 and 12 = 3.08 μM; 0.1 μg/μl permethrin = 2.55 μM

TABLE 3
Activity of compounds 11 and 12 against adults
of ORL and PR strains of Ae. aegypti

| | | Percent Mortality^{a} | | |
|---|---|---|---|---|
| | | Compound concentration (%)^{b} | | |
| Compound | Strain | 6.25 | 3.125 | 1.56 |
| 11 | ORL | 24.2 ± 10.1 | 3.3 ± 5.8 | — |
| | PR | 30 ± 26.5 | 23.3 ± 20.8 | 10 ± 10 |
| 12 | ORL | 20 ± 10 | 3.3 ± 5.8 | 3.3 ± 5.8 |
| | PR | 33.3 ± 30.6 | 12.1 ± 21 | 20 ± 20 |

| | | |
|---|---|---|
| Untreated | ORL | 0 |
| | PR | 3.3 ± 5.8 |
| Acetone | ORL | 3.3 ± 5.8 |
| | PR | 6.7 ± 2.9 |
| Permethrin | ORL | 100 |
| | PR | 85 ± 10 |

^{a}Values are percent mortality ± standard deviation for adult mosquitos after 24 h
^{b}6.25, 3.125, and 1.56% of compounds 11 and 12 = 192.65, 96.32, and 48.08 μM, respectively; 6.25% permethrin = 159.72 mM

Both of compounds 11 and 12 were highly effective against larvae of ORL and PR strains. Compound 11 showed a slightly better activity than 12, and showed the same activity as permethrin at 0.1 μg/μl concentration, indicating a (E)-configuration may be preferable for larvicidal activity. Both compounds were somewhat effective against adult mosquitoes of both strains, but had low effectiveness as compared to the permethrin.

### EXAMPLE 3

### Nematicidal Activity: Meloidogyne incognita

The synthesized 3,5-dimethoxystilbene analogs were evaluated for activity against nematodes, using the plant-parasitic nematode Meloidogyne incognita as a model.

Two types of assays were used to investigate activity against M. incognita: i) eggs were immersed in solutions of the test compounds, and ii) second-stage juveniles (J2) previously hatched from eggs were immersed in the test solutions. These assays were conducted with procedures similar to those in Meyer, et al. (J. Nematol. 2006, 38, 333). M. incognita race 1 (originally isolated in 2013 from a field in Maryland) was grown in the greenhouse on pepper (Capsicum annuum L.) cultivar PA-136. Egg masses were hand-picked from plant roots, rinsed three times with sterile distilled H₂O, and agitated in 0.6% NaClO for 3.5 minutes to separate and surface-sterilize eggs. The eggs were rinsed in sterile distilled H₂O and stored overnight at 4° C. before use in egg immersion assays. To collect J2 for direct immersion into the test compounds, sterilized eggs were placed into a hatching chamber made with a Spectra/Mesh Nylon Filter (openings 25 μm in diameter; Spectrum Laboratories Inc., Rancho Dominguez, Calif., USA) in an autoclaved dish. J2 that passed through the filter within 3 days were used immediately for assays.

The assays were conducted in 96-well polystyrene plates. For the assays with immersed eggs, each well received approximately 50 eggs in 35 μl of sterile deionized water (SDW). For assays with previously hatched J2, a suspension of approximately 50 J2 in 35 μl of SDW was placed into each well. Each well then received 165 μl of treatment or control. The solvent used to dissolve the test chemicals was a 1:1:1 mixture referred to herein as CTD: equal parts Cremophor® EL Castor Oil (BASF Corporation, Vandalia, Ill., USA), Tween 80® (Sigma-Aldrich, St. Louis, Mo., USA), and DMSO (Sigma-Aldrich). A high and low rate of each compound was tested. After addition to the nematode suspensions in the wells, the low and high rates were 83.3 and 166.7 μg/ml of each test compound, respectively, dissolved in 0.5% and 1.0% of each of the three combined solvents. Controls were CTD equivalent to the low and high rates, and SDW. Because of the large number of treatments, the test compounds were divided into two groups for the egg immersion assays. The compounds tested in the egg immersion Assay 1 were 3, 6a, 6e, 6f, 6g, 6n, 7a, 7c, 7e, 7f, 7n, 11, 12, 16, and 17. The compounds tested in the egg immersion Assay 2 were 6b, 6h, 6i, 6j, 6k, 6l , 7b, 7g, 7h, 7i, 7j, 7k, 7l, and 7m (data not shown). Test compounds used for assays with previously hatched J2 were selected based on results with the egg assays. The five compounds tested were 6b, 6f, 6g, 6i, and 7k, high and low rates. Assays were then repeated with 6g and 7k, high rates (data shown in Table 4 below),

Each polystyrene culture plate was covered with a plastic adhesive sheet (Excel Scientific, Inc., Victorville, Calif., USA) and incubated at 25° C. Ten wells (water controls) or five wells (all other treatments) were used per treatment in each assay. In egg immersion assays, total numbers of hatched J2, and numbers of mobile and immobile J2, were counted after 2 and 7 days incubation in the test compounds. In the first assay with previously hatched J2, the numbers of mobile and immobile J2 were counted after 1 and 2 days incubation, the treatments removed and replaced with a SDW rinse, and 2 days later the mobile vs. immobile J2 counted again. In assays with 6g and 7k, high rates and counts were made on Days 1, 2, 4, and 6, without a SDW rinse.

Data from the M. incognita assays were analyzed with the statistical package JMP 11.2.0 (SAS Institute, Cary, N.C., USA). Differences among numbers of hatched J2 in each treatment, and among percentage of mobile J2 per treatment (number mobile J2/total J2×100), were determined by the ANOVA, and means were compared using Tukey Kramer's adjustment for multiple comparisons (P<0.05). For percent mobile J2 on on Day 2 in egg immersion Assays 1 and 2, and Day 4 with the five compounds in the J2 immersion assay, data were log10 (x+1)-transformed before analysis. Data presented are nontransformed means.

TABLE 4
Activity of analog compounds against M. incognita
previously hatched J2 immersed in treatment solutions

| | | Percent | Percent |
|---|---|---|---|
| No. of | | mobile J2, | mobile J2, |
| days | Treatment^{a} | assay 1^{b} | assay 2^{b} |
| 2 | H₂O | 87.8% | 97.7% |
| | CTD low | 82.0% | 100.0% |
| | CTD high | 75.1% | 100.0% |
| | 6i low | NT^{c} | 71.0% |
| | 6i high | NT | 100.0% |
| | 7k low | NT | 92.5% |
| | 7k high | NT | 72.2% |
| 7 | H₂O | 94.0% | 96.1% |
| | CTD low | 89.8% | 96.7% |
| | CTD high | 90.2% | 94.1% |
| | 6f low | 93.9% | NT |
| | 6f high | 71.3% | NT |
| | 6g low | 89.2% | NT |
| | 6g high | 74.6% | NT |
| | 6i low | NT | 87.7% |
| | 6i high | NT | 93.2% |

^{a}6f low = 214.64 μM, high = 429.54 μM; 6g low = 250.87 μM, high = 502.05 μM; 6i low = 258.65 μM, high = 517.62 μM; 7k low = 265.16 μM, high = 530.64 μM
^{b}means are comparable within columns; data between assay 1 and assay 2 are not comparable
^{c}NT = not tested in this assay

None of the tested compounds suppressed hatch of M. incognita eggs. There was some suppressive effect on mobility in J2 that had hatched from eggs immersed in solutions of 6f, 6g, 6i, and 7k (Table 4). However, when J2 previously hatched in water were immersed directly into these same compounds or 6b, only 6g and 7k had an effect on J2 mobility.

### EXAMPLE 4

### Fungicidal Activity

The analogs were tested for activity against Colletotrichum acutatum SIMMONDS, C. fragariae BROOKS, and C. gloeosporioides (PENZ) PENZ & SACC. in Penz., using a direct bioautography method as previously described by Tabanca et al. (Nat. Prod. Commun. 2008, 3, 1073) and Wang, et al. (J. Agric. Food Chem. 2013, 61, 4551). Technical grade commercial fungicide standards benomyl, cyprodinil, azoxystrobin, and captan were used at 0.9-1.61 μg/μl concentrations in 95% EtOH. After sample application, each TLC plate was subsequently sprayed with a spore suspension (3.0×10⁵ spores/ml) of the fungus of interest and incubated in a moisture chamber for 4 days at 26° C. with a 12 hour photoperiod. Clear zones of fungal growth inhibition on the TLC plate indicated the presence of antifungal constituents in each extract or pure compound. The test compounds were prepared at 2 mM soln. in 95% EtOH, and 4 μl were spotted on the plates (2 μl of the positive controls).

When evaluated for activity against the fungi, compounds 6a, 7a, and 7e had a strong inhibitory effect on Colletotrichum species. See Table 5 below:

TABLE 5
Activity of analog compounds against Colletotrichum sp.

| Treatment^{a} | C. acutatum^{b} | C. fragariae^{b} | C. gloeosporioides^{b} |
|---|---|---|---|
| 6a | 5 | 5 | 5.5 |
| 7a | 8 | 6.5 | 7 |
| 7e | 6 | 6 | 6 |
| Azoxystrobin | 15 | 17.5 | 17.5 |
| Benomyl | 17 | 20 | 20 |
| Captan | 11.5 | 18.5 | 18.5 |
| Cyprodinil | 18 | 21.5 | 21.5 |

^{a}Concentrations of all treatments is 2 mM. Thus, there is 2.03 μg of 6a and 7a in 4 μl, 2.17 μg of 7e in 4 μl, and 1.61 μg of Azoxystrobin, 1.16 μg of Benomyl, 1.20 μg of Captan, and 0.90 μg of Cyprodinil, each, in 2 μl
^{b}Values are zones of inhibition (in mm), average of 2 assays

### EXAMPLE 5

### Antimicrobial Activity

The activity of the analogs against some human pathogens was also explored.

All organisms were obtained from the American Type Culture Collection (ATCC; Manassas, Va., USA) and included the fungi Cryptococcus neoformans ATCC 90113 and the bacteria Staphylococcus aureus ATCC 29213, methicillin-resistant S. aureus ATCC 33591 (MRSA), and Mycobacterium intracellulare ATCC 23068. All organisms were tested using modified versions of the CLSI (formerly NCCLS) methods. For all organisms excluding My. intracellulare, optical density was used to monitor growth. Medium supplemented with 5% AlamarBlue® (BioSource International, Camarillo, Calif., USA) was utilized for growth detection of My. intracellulare. Samples (dissolved in DMSO) were serially diluted in 20% DMSO/saline and transferred (10 μl) in duplicate to 96-well flat-bottom microplates. Inocula were prepared by correcting the OD630 of microbe suspensions in Sabouraud Dextrose for Cr. neoformans, cation-adjusted Mueller-Hinton (Difco) at pH 7.3 for Staphylococcus spp., and 5% AlamarBlue® (BioSource International) in Middlebrook 7H9 broth with OADC enrichment, pH=7.0 for My. intracellulare, to afford an assay volume of 200 μl and final target inocula of: Cr. neoformans.: b 1.5×10³, My. intracellulare: 2.0×10⁶, and Staphylococcus spp.,: 5.0×10⁵ CFU/ml. Final sample test concentrations were 1/100th the DMSO stock concentration. Drug controls (Ciprofloxacin (ICN Biomedicals, Ohio, USA) for bacteria and Amphotericin B (ICN Biomedicals) for fungi) were included in each assay. All organisms were read at either 530 nm using the Biotek Powerwave XS plate reader (Bio-Tek Instruments, VT) or 544ex1590em (My. intracellulare) using the Polarstar Galaxy Plate Reader (BMG LabTechnologies, Germany) before and after incubation: Staphylococcus spp. at 35° C. for 16-20 h, Cr. neoformans at 35° C. for 70-74 h, and My. intracellulare at 37° C. and 10% CO2 for 70-74 h. IC₅₀ Values (concentrations that afford 50% inhibition relative to controls) werecalculated using XLfit 4.2 software (IDBS, Alameda, Calif.) using fit model 201. Results are shown in Table 6 below:

TABLE 6
Activity of analog compounds against human pathogens

| | Cr. neoformans | | S. aureus | | MRSA^{a} | | My. intracellulare | |
|---|---|---|---|---|---|---|---|---|
| Treatment | IC₅₀^{b} | MIC^{b} | IC₅₀ | MIC | IC₅₀ | MIC | IC₅₀ | MIC |
| 11 | 13.63 | 30.84 | 13.11 | 30.84 | 9.10 | 15.42 | 47.17 | 61.68 |
| 12 | 41.77 | 61.68 | 17.03 | 30.84 | 13.02 | 30.84 | 50.40 | 61.68 |
| 16 | 5.22 | 9.25 | 20.43 | 74.04 | 43.72 | 74.04 | 33.98 | 74.04 |
| Amphotericin B | 0.698 | 1.353 | — | — | — | — | — | — |
| Ciprofloxacin | — | — | 0.383 | 1.509 | 0.356 | 1.509 | 0.815 | 1.509 |

^{a}MRSA = methicillin-resistant S. aureus
^{b}IC₅₀ and MIC units are μM

Compounds 11, 12, and 16 showed moderate inhibition of Cr. neoformans (IC₅₀=13.63, 41.77, and 5.22 μM, respectively), My. intracellulare (IC₅₀=47.17, 50.40, and 33.98 μM, respectively), S. aureus (IC₅₀=13.11, 17.03, and 20.43 μM, respectively), and methicillin-resistant S. aureus (IC₅₀=9.10, 13.02, and 43.72 μM, respectively). Compounds 11, 12, and 16 had MIC (minimum inhibitory concentration) values 42-344 times less than that of 3,5-dimethoxystilbene. This suggests that addition of OH at C(4′) enhances activity. The results indicate that 3,5-MeO and 4′-OH, the common structural features of 11, 12 and 16, must be kept intact for activity. The addition of a prenyl moiety at C(3′) appears to further nhance inhibitory activity against S. aureus and methicillin-resistant S. aureus, as observed from the relative MICS of compounds 11 and 16.

Studies on stilbenes against the Mycobacterium species are scanty, and most report the absence of or insignificant activity. This is the first report of stilbenes having activity against My. intracellulare. Thus, finding compounds 11, 12 and 16 as inhibitory, albeit moderately, is quite stimulating.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A composition comprising a stilbene analog, wherein the stilbene analog is one of (Z)-1,3-dimethoxy-2-methyl-5-styrylbenzene (compound 7a), (E)-1,3-dimethoxy-5-(3-methoxystyryl)-2-methylbenzene (compound 6b), (Z)-1,3-dimethoxy-5-(3-methoxystyryl)-2-methylbenzene (compound 7b), (E)-1,3-dimethoxy-5-(4-methoxystyryl)-2-methylbenzene (compound 6c), (Z)-1,3-dimethoxy-5-(4-methoxystyryl)-2-methylbenzene (compound 7c), (E)-1,3-dimethoxy-5-(4-nitrostyryl)-2-methylbenzene (compound 6d), (Z)-1,3-dimethoxy-5-(4-nitrostyryl)-2-methylbenzene (compound 7d), (E)-1,3-dimethoxy-5-(4-fluorostyryl)-2-methylbenzene (compound 6e), (Z)-1,3-dimethoxy-5-(4-fluorostyryl)-2-methylbenzene (compound 7e), (E)-1,3-dimethoxy-5-(4-chlorostyryl)-2-methylbenzene (compound 6f), (Z)-1,3-dimethoxy-5-(4-chlorostyryl)-2-methylbenzene (compound 7f), (E)-1,3-dimethoxy-5-(4-bromostyryl)-2-methylbenzene (compound 6g), (Z)-1,3-dimethoxy-5-(4-bromostyryl)-2-methylbenzene (compound 7g), (E)-1,3-dimethoxy-5-(4-trifluoromethylstyryl)-2-methylbenzene (compound 6h), (Z)-1,3-dimethoxy-5-(4-trifluoromethylstyryl)-2-methylbenzene (compound 7h), (E)-1,3-dimethoxy-5-(3,4-dichlorostyryl)-2-methylbenzene (compound 6i), (Z)-1,3-dimethoxy-5-(3,4-dichlorostyryl)-2-methylbenzene (compound 7i), (E)-1,3-dimethoxy-5-(2,4-dimethoxystyryl)-2-methylbenzene (compound 6j), (Z)-1,3-dimethoxy-5-(2,4-dimethoxystyryl)-2-methylbenzene (compound 7j), (E)-1,3-dimethoxy-5-(3,4-dimethoxystyryl)-2-methylbenzene (compound 6k), (Z)-1,3-dimethoxy-5-(3,4-dimethoxystyryl)-2-methylbenzene (compound 7k), (E)-1,3-dimethoxy-5-(3,5-dimethoxystyryl)-2-methylbenzene (compound 6l), (Z)-1,3-dimethoxy-5-(3,5-dimethoxystyryl)-2-methylbenzene (compound 7l), (E)-1,3-dimethoxy-5-(2,6-dimethoxy-4-methylstyryl)-2-methylbenzene (compound 6m), (Z)-1,3-dimethoxy-5-(2,6-dimethoxy-4-methylstyryl)-2-methylbenzene (compound 7m), (Z)-4-(3,5-dimethoxystyryl)-2-(3-methylbut-2-en-1-yl)phenol (compound 12), (E)-4-(3,5-dimethoxy-4-methylstyryl)phenol (compound 16), and (Z)-4-(3,5-dimethoxy-4-methylstyryl)phenol (compound 15).

2. The composition of claim 1, wherein the composition has biological activity.

3. The composition of claim 2, wherein the biological activity is at least one of insecticidal activity, larvicidal activity, fungicidal activity, nematicidal activity, antimicrobial activity, antibiotic activity, and bactericidal activity.

4. The composition of claim 1, further comprising a carrier.

5. The composition of claim 1, wherein the stilbene analog is one of compounds 7a, 7e, 6f, 6g, 6i, 7k, 12, 15, and 16.

6. A method of producing the stilbene analog of claim 1, the method comprising:
reacting a benzene-containing phosphonium salt with a benzaldehyde analog in the presence of butyllithium, and
thereby producing the stilbene analog.

7. A method of treating for mosquitos, the method comprising:
applying an effective amount of a pesticide to a plant or area,
wherein the pesticide is an analog of 3,5-dimethoxystilbene.

8. The method of treating for mosquitos according to claim 7, wherein the pesticide has a prenyl group substitution in the 3-position of the non-dimethoxy-substituted benzene ring.

9. The method of treating for mosquitos according to claim 8, wherein the pesticide is one of (E)-4-(3,5-dimethoxystyryl)-2-(3-methylbut-2-en-1-yl)phenol (compound 11) and (Z)-4-(3,5-dimethoxystyryl)-2-(3-methylbut-2-en-1-yl)phenol (compound 12).

10. The method of treating for mosquitos according to claim 7, wherein the mosquitos are Aedes sp.

11. The method of treating for mosquitos according to claim 10, wherein the mosquitos are Ae. aegypti.

12. A method of treating for nematodes, the method comprising:
applying an effective amount of a nematicide to a plant or area,
wherein the nematicide is an analog of 3,5-dimethoxystilbene.

13. The method of treating for nematodes according to claim 12, wherein the nematicide is one of (E)-1,3-dimethoxy-5-(4-chlorostyryl)-2-methylbenzene (compound 6f), (E)-1,3-dimethoxy-5-(4-bromostyryl)-2-methylbenzene (compound 6g), (E)-1,3-dimethoxy-5-(3,4-dichlorostyryl)-2-methylbenzene (compound 6i), (Z)-1,3-dimethoxy-5-(3,4-dimethoxystyryl)-2-methylbenzene (compound 7k), and (E)-5-(4-hydroxystyryl)-2-methylbenzene-1,3-diol (compound 17).

14. The method of treating for nematodes according to claim 12, wherein the nematodes are Meloidogyne sp.

15. The method of treating for nematodes according to claim 14, wherein the nematodes are M. incognita.

16. A method of treating for fungi, the method comprising:
applying an effective amount of a fungicide to a plant or area,
wherein the fungicide is an analog of 3,5-dimethoxystilbene.

17. The method of treating for fungi according to claim 16, wherein the fungicide is one of (E)-1,3-dimethoxy-2-methyl-5-styrylbenzene (compound 6a), (Z)-1,3-dimethoxy-2-methyl-5-styrylbenzene (compound 7a), and (Z)-1,3-dimethoxy-5-(4-fluorostyryl)-2-methylbenzene (compound 7e).

18. The method of treating for fungi according to claim 16, wherein the fungi are Colletotrichum sp.

19. The method of treating for fungi according to claim 18, wherein the fungi are one of C. acutatum, C. fragariae, and C. gloeosporioides.

20. A method of treating for a human pathogen, the method comprising:
providing an effective amount of an active compound,
wherein the active compound is an analog of 3,5-dimethoxystilbene, and
wherein the active compound has a hydroxy group substitution in the 4-position of the non-dimethoxy-substituted benzene ring.

21. The method of treating for a human pathogen of claim 20, wherein the active compound has a prenyl group substitution in the 3-position of the non-dimethoxy-substituted benzene ring.

22. The method of treating for a human pathogen of claim 20, wherein the active compound is one of (E)-4-(3,5-dimethoxystyryl)-2-(3-methylbut-2-en-1-yl)phenol (compound 11), (Z)-4-(3,5-dimethoxystyryl)-2-(3-methylbut-2-en-1-yl)phenol (compound 12), (Z)-4-(3,5-dimethoxy-4-methylstyryl)phenol (compound 15), (E)-4-(3,5-dimethoxy-4-methylstyryl)phenol (compound 16), and (E)-5-(4-hydroxystyryl)-2-methylbenzene-1,3-diol (compound 17).

23. The method of treating for a human pathogen of claim 20, wherein the human pathogen is one of Cryptococcus neoformans, Staphylococcus aureus, and Mycobacterium intracellulare.
